(19) United States
(12) Patent Application Publication
(10) Pub. No.: US 2024/0321440 A1
(43) Pub. Date: Sep. 26, 2024

(54) AUTONOMOUS MEDICAL KIT TRACKING DEVICE THAT WITHSTANDS AUTOCLAVE

(71) Applicant: Q Med Innovations, Inc., Middletown, RI (US)

(72) Inventors: Shelby Kornbluth, Foxboro, MA (US); Victor Nunes, Middletown, RI (US); James Calvin, Attleboro, MA (US)

(73) Assignee: Q Med Innovations, Inc., Middletown, RI (US)

(21) Appl. No.: 18/186,263

(22) Filed: Mar. 20, 2023

(51) Int. Cl. G16H 40/60; G06F 1/28

(52) U.S. Cl. CPC G16H 40/60; G06F 1/28

(57) ABSTRACT

The present disclosure is directed to an instrument kit tracking system that includes a housing that has tracking electronics that is contained in either a compartment or wall of the medical instrument kit or in a standalone unit that is affixed to a medical instrument kit to contain and protect an electronic device from external temperatures up to 132° C., and higher; where the medical instrument kit contains at least one medical instrument. The internal temperature of the instrument kit tracking system is maintained such that the electronic device contained therein is operable when the external temperature is up to 132° C., and higher. Hardware system architecture and integrated power management intelligently controls power consumption of electronic components for electronic component safety and integrity, efficiency and long term battery life of the device.

### CROSS REFERENCE TO RELATED APPLICATIONS

N/A

### BACKGROUND OF THE INVENTION

The present disclosure generally relates to the medical industry, specifically to medical instrument kits as well as the object tracking industry, namely tracking systems for medical instrument kits.

In the medical industry, current instrument kits suffer from the inability to properly track such instrument kits once they are deployed to the sales force network or hospitals. These instrument kits are often assets of the original equipment manufacturers (OEM) and often placed on loan to a hospital to support a surgical procedure. One issue with such kits is, once they are out of the OEM's control, the sets are often misplaced, lost, or simply contained within the sales network without further visibility by the OEM. These lost, misplaced, or otherwise disposed kits come at an extreme cost to the OEM's each year.

Additionally, there is a concern in the industry and by the FDA as to how instruments are being managed. Such management can include the calibration, repair, or ultimately replacement of the instruments once they are deployed to support surgery. Current instrument kits fail to provide a way of tracking whether a kit has been in one or a hundred surgeries. As such, the prior art systems fail to provide the OEM, hospitals, or other users the current lifecycle of the kits or the instruments contained therein.

Solutions to address known problems in the industry have been primarily software solution, RFID which required human intervention or Bluetooth which requires infrastructure. For example, there have been attempts to track surgical assets with software applications and phone apps to facilitate the management of the location of these sets and replenishment of implant or instrument inventory. These efforts are inadequate and suffer from inaccuracy and lack of integrity because these tools require manual input and many times data is corrupted due to the lack of input given to the systems on a timely basis. Thus, these attempts in the prior art to address these concerns have not been successful.

There is also a need for such medical instrument device tracking devices to travel with the medical instrument kit as much as possible thereby to provide comprehensive tracking of the location of that medical kit. This not only includes when the medical instrument kit is in an operating room or in storage but also when the kit is being sterilized, such as by an autoclave, which places the tracking device under very high heat and pressure. However, such tracking devices include very sensitive electronics to carry out the tracking and location reporting so these devices are susceptible to failure and degradation of battery life. Therefore, there is a need for better power management and heat control in such tracking devices.

In addition, the present invention relates to devices that can be used in transport, company facility, filed inventory location, a hospital or ambulatory surgery center setting.

### SUMMARY OF THE INVENTION

The device of the present invention has particular applicability and use in transport, company facility, field inventory location, a hospital or ambulatory surgery center settings where the device may be attach to or inside instrument kits, such reusable instrument kits that are steam sterilized in an autoclave.

The instrument kit tracking system and methods of the present invention can provide the ability to monitor and report on a daily basis the exact location of each kit without human interaction. Moreover, the instrument kit tracking system and method of the present invention can record and transmit, within the same daily schedule, all date stamps, locations, and number of surgeries the kit has been involved in. A cloud-based system can be included to capture data on a daily basis and report out to the user or manager the particular status of each deployed kit. The cloud-based system can additionally alert end users of any kit that may have reached a predefined limitation on the number of surgeries performed so that the kit can be returned and instruments replaced or calibrated before going back out for deployment. The instrument kit tracking system and method of the present invention offers greater efficiency in inventory management, deployment, accountability of the assets, automatic annual inventory auditing, complete compliance to instrument usage and documentation, and ease of inventory recovery during product recalls.

The device, system, and method of the present invention addresses the foregoing concerns and addresses the shortcomings in prior art devices, systems, and methods.

The present invention addresses the concerns in the industry by providing an instrument kit tracking system that includes a housing that has tracking electronics that is either attached to a medical instrument kit or contained in such a medical instrument kit to be tracked. The tracking device of the present invention can withstand high external temperatures, such as 132° C., and high pressure, such as 28 psi. The electronics contained therein is operable or maintains their integrity when the external temperature and pressure are at those high levels. The hardware system architecture and integrated power management intelligently controls power consumption of electronic components for electronic component safety and integrity, efficiency and long term battery life of the device.

Thus, the present invention addresses the foregoing needs in the industry to provide not only continuous tracking of surgical, or medical kits, but also with withstand high temperature and pressure sterilization processes, such in an autoclave for more comprehensive medical instrument kit tracking.

### BRIEF DESCRIPTION OF THE DRAWING FIGURES

The novel features that are characteristic of the present disclosure are set forth in the appended claims. However, the disclosure's preferred embodiments, together with further objects and attendant advantages, will be best understood by reference to the following detailed description taken in connection with the accompanying drawings in which:

FIG. 1 is a top view of the rigid flex circuit and electronics of the tracking device of the present invention in a flat, unfolded condition;

FIG. 2 is a perspective view of the rigid flex circuit and electronics of the tracking device of the present invention in a folded, partially assembled condition;

FIG. 3 is a perspective view of the rigid flex circuit and electronics of the tracking device of the present invention in a folded, fully assembled condition with phase change material pillow installed in the device;

FIG. 4 is a top view of heat dissipation member in the form of a phase change material pillow that is packed inside the device of the present invention, shown in FIG. 3, for additional cooling capability;

FIG. 5 is a diagram of the power management that is employed by the present invention;

FIG. 6 is a block diagram showing the hardware system architecture of the device of the present invention;

FIG. 7 is a chart showing the internal board temperature of the device of the present invention over time during an autoclave cycle;

FIG. 8 is a chart showing the results of the present invention and its effectiveness in controlling the temperature of the device so it may safely withstand an autoclave sterilization process; and

FIG. 9 is a perspective view of the tracking device of the present invention attached to a medical instrument kit.

### DESCRIPTION OF THE INVENTION

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like proximal, distal, top, bottom, up, or down are used, they are not intended to limit the systems, devices, and methods disclosed herein. A person skilled in the art will recognize that these terms are merely relative to the system and device being discussed and are not universal.

In the prior art, as stated above, it is well known to bundle common surgical instruments which are required for a particular type of surgical procedure together in specialized kits. There are numerous health and safety requirements for these kits including tight inventorying and sterilization. Current systems rely on manual recording of the particular kits by serial number, the number of uses of each kit, and the locations of such uses. However, current systems lack the ability to automatically track the location and uses of each kit, notify end users of that data, and continue to provide that data even during high temperature sterilization processes.

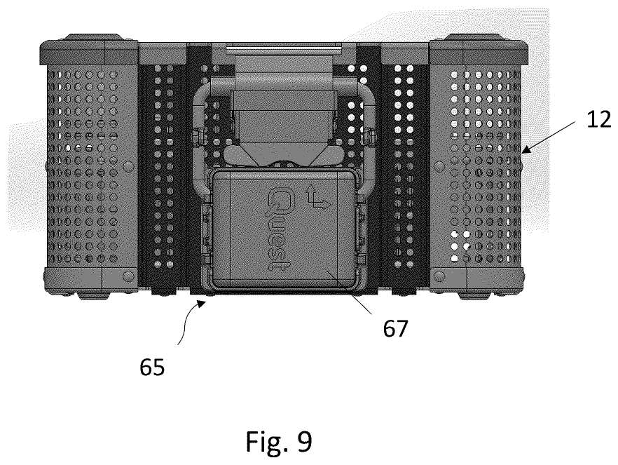

The instrument kit tracking system 65 of the present invention can be placed on or inside an instrument kit 12, representationally shown in FIG. 9 as attached to a medical instrument kit 12, that is then steam sterilized at 132° C. and 28 psi, for example, using an autoclave (not shown). Autoclaves are so well known in the art, they need not be discussed in detail herein. The present instrument kit tracking system 65 provides the ability to monitor surgical events and report on these events and location on a daily basis without human interaction.

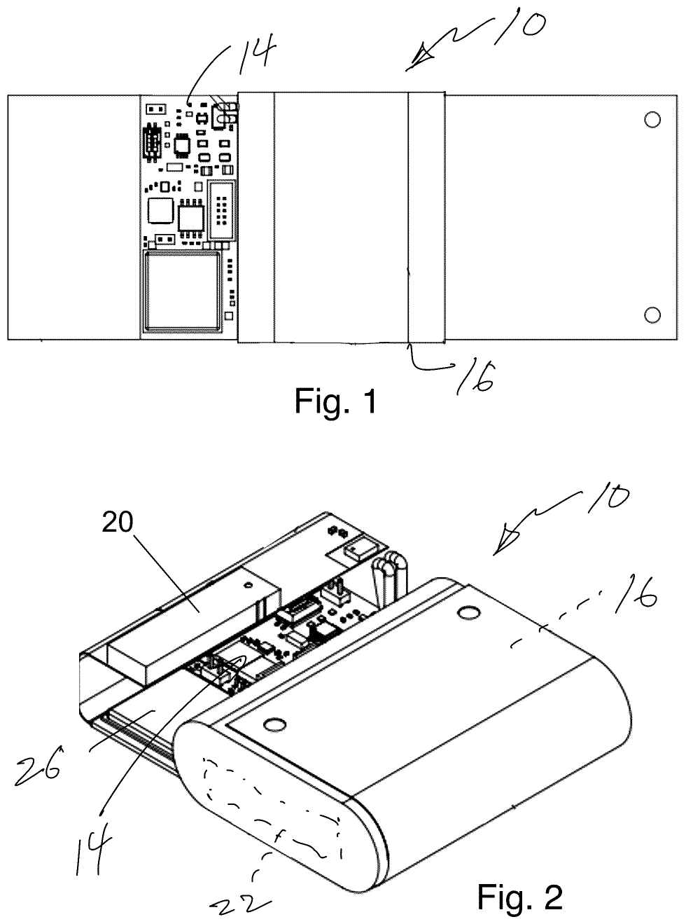

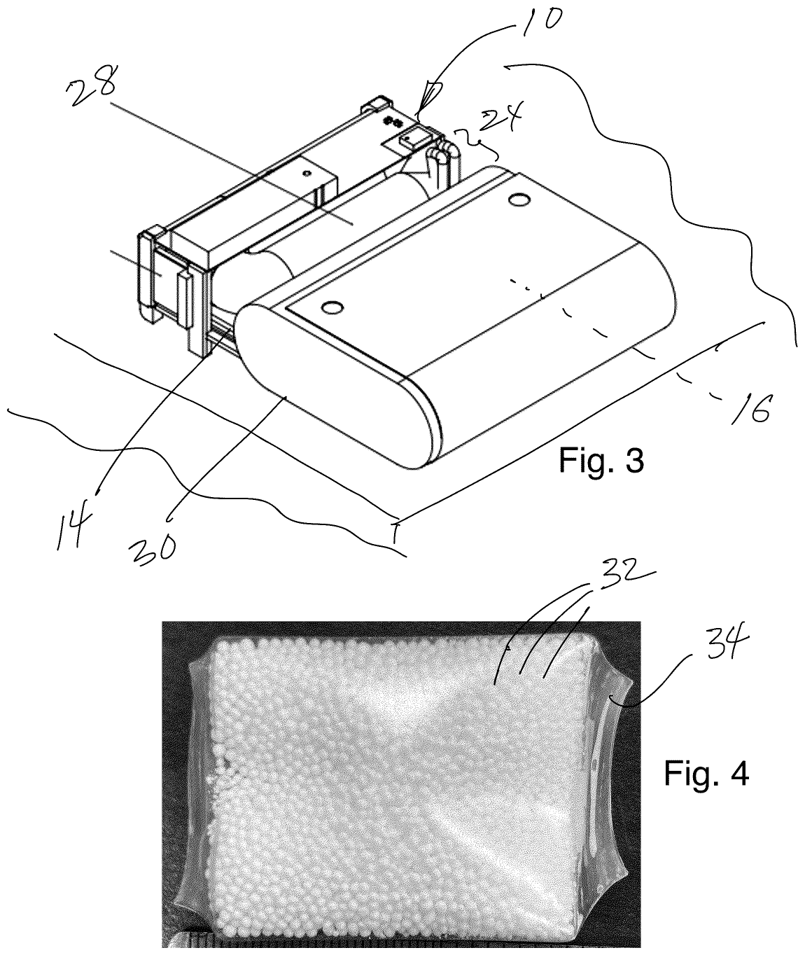

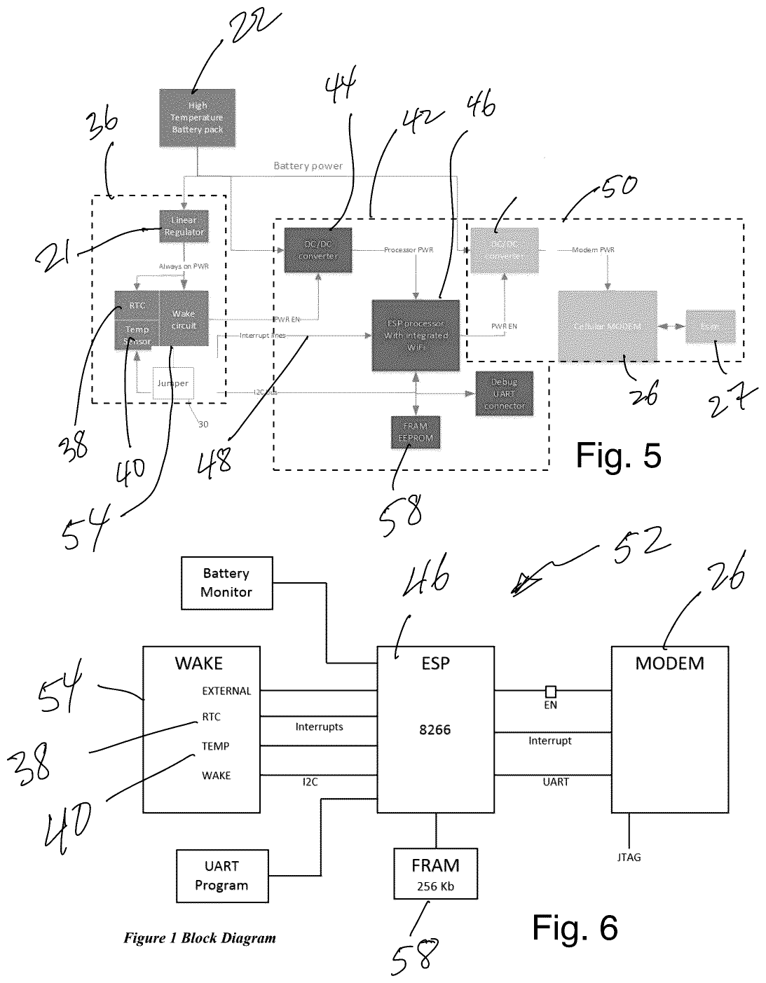

Referring now to FIGS. 1-4 and 9, details of the construction of the tracking system 65 of the present invention are shown. First, the device 65 must be able to be attached to surgical medical instrument kit 12 without interfering with the kit's function. The present invention 65 achieves this due its small size. However, this small size itself presents a problem to the electronics 14 due the large ground plane required by the cellular antenna 20 that is electrically interconnected to the cellular modem 26. This is addressed through the use of a rigid-flex circuit 10 that is folded into the sealed housing 67. This solves the small compact size requirement and still provides a large enough ground plane for the cellular antenna 20.

The tracking device and system 65 of the present invention is smaller than a traditional printed circuit board assembly (“PCBA”) while still maintaining a large enough ground plane to maximize performance of the antenna. Using the rigid-flex circuit 10, the components are assembled on the rigid section 14 of the rigid-flex circuit, the flex circuit 16 wraps around the batteries 22 and provides a large enough ground plane for the antenna 20. FIG. 1 shows the rigid-flex circuit 10 of the present invention in an unfolded flat condition. FIG. 2 shows the rigid-flex circuit 10 of the present invention in a folded condition. The rigid-flex circuit 10 of the present invention provides a cooling device receiving region 24. FIG. 3 shows the installation of an optional insulation material 28 in the cooling device receiving region 24 to protect the most sensitive electronic components 14 from being exposed to high heat. The fully assembled rigid-flex circuit 10 is installed into the housing 67. Such an optional cooling material 32 is shown in FIG. 4, which is preferably in the form of an insulation pillow made of an organic phase change material that is inside a tube 34 sealed on both ends. The addition of the phase change material 32 advantageously drops the temperature inside the device 10 of the present invention by several degrees, such as seven degrees.

The instrument kit tracking system 65 of the present invention transmits its data to the cloud daily or anytime it detects an event, such as a decontamination or autoclave cycle. The present instrument kit tracking system 65 transmits such data preferably wirelessly, such as via a cellular modem 26, which may be LTE-M or other network or protocol.

The instrument kit tracking system of the present invention overcomes many of the obstacles and disadvantages found in the prior art. For example, the system 65 of the present invention can withstand high pressure steam, such as 28 psi high pressure steam that is delivered by an autoclave. It overcomes this concern via a hermetically sealed box 30. This does not allow any moisture (steam or liquid water) into the housing 67 of the instrument kit tracking system 65. Also, the system 65 of the present invention can withstand the high temperatures associated with an autoclave, such as temperatures in the range of 132 degrees C.

To carry this out, the system 65 of the present invention does this by use of high temperature batteries 22, which are typically rated at 150 degrees C. The phase change material 32, 34 is used to blunt the peak heat from the autoclave by melting and reducing the peak temperature thru storing heat and releasing it later in time when removed from the autoclave.

Referring now to FIG. 5, the system 65 of the present invention and how it optimizes power management is described. The power management is broken into three zones. The first zone 36 is the “always ON” zone. This is never is OFF and is supplied power via a linear regulator 21 connected to the battery 22. This zone 36 has a real time clock (RTC) 38, and a temperature sensor 40. The first zone 36 controls the power of second zone 42, which is the processor power zone. It carries this out by using alarm and interrupt lines from the RTC 38 and temperature sensor 40 through an Oring circuit to enable a DC/DC converter 44 that provides power to the processor section 42, namely zone 2.

A jumper 30 is also provided to force the second zone ON so the RTC and temperature sensors can be initialized by the processor (through the I2C bus) at start up. The first zone 36 (always ON zone) then turns on the processor 46 at a specified time and/or at a specified temperature or temperatures. It also connects interrupt lines 48 to the general purpose input/output (“GPIO”) pins on the processor. As a result, the processor 46 can determine which device (RTC 38 or temperature sensor 40) it powered on.

The third and final zone 50 is the modem power zone. This third power zone 50 is controlled by the second zone 42 (processor zone). The third zone 50 is only powered when the processor 46 wants to make a cellular call for data transmission, and the like. The processor 46 does this by using a GPIO pin to enable a DC/DC converter 44 providing power to the modem 26. FIG. 5 illustrates these three zones 36, 42, and 50 where the modem power zone 50 cannot be ON if the second zone 42 is not ON.

The firmware logic uses an autoclave profile to so that zone 2 42 and zone 3 50 are never ON or operating in an autoclave and when temperatures are above a specific set point. The foregoing parameters are preferably configurable by software. All the components in zone 1 36 are rated for a die temperature of 150 degrees C. and are powered by minimum on voltage to minimize self-heating.

Tests were performed to ensure all zone 1 components could operate at up to 140 degrees C. All the components in zone 2 42 and 3 50 are preferably rated for a storage temperature of 150 degrees C. They are powered OFF when the temperature exceeds a configurable set point, typically 105 degrees C. They do not turn back ON until the temperature returns to below a lower set point, typically 85 degrees C., or the like, to provide thermal hysteresis.

With the hardware configuration and power management, it is possible for the device and system 10 to run on the same battery 22 for several years, such as five years. This is achieved a through a combination of high amperage hour batteries 22 and unique power management, as disclosed herein. The device 10 of the present invention is only ON for typically 30 seconds a day. For the remaining time, only standby power is used (in zone 1 36 of the power zones, as in FIG. 5), which consumes very little power, such as only a few microamps of current. Thus, the device 10 of the present invention employs a power management system and method that can achieve 5 year operating life, or more.

Turning now to FIG. 6, hardware system architecture 52 is shown in detail, which carries out the power management system and method of the present invention. A logical view of the architecture can be seen, which has four general functional sections.

First, the WAKE section 54 includes a temperature sensor 56 and a real time clock 58, which enables the power to the processor 46, such as an event stream processor (ESP functional section) based on temperature or time.

Second, the ESP 46 is provided, which is always powered OFF until it acts on the waking source to scan for Wi-Fi (which may also be provided for transmission of data), temperature and/or battery voltage.

Third, a modem 26 is provided, which controls communication to a host whereby its power is controlled by the ESP section 46.

Fourth, storage 58 is provided preferably in the form of non-volatile memory, which may be ferroelectric random access memory (FRAM), or the like.

Functionally, the architecture and hardware control are what is enabled (or awake) via temperature and the real time clock (RTC) 38 to conserve power and protect the electronics 14 of the device 10. Details of such control are outlined below.

More specifically, as far as operation and control of the device 10, the WAKE section 54 preferably measures temperature at a configured time interval, such as every 5 minutes. If the temperature reaches T1, such as 60° C., it wakes up the ESP 46 to start recording the temperature at the configured time interval, such as 5 minutes. The ESP 46 enables STORAGE 58 where the temperature data is stored. Once the temperature reaches T2, such as 110 degrees C., it disables the RTC 38 and powers down. This is to insure the components are not operating at temperatures above 110 degrees C. since many of the components have a maximum operating temperature between 115 and 120 degrees C. but have a storage temperature of up to 150 degrees C. Once the temperature drops to a safe temperature T3, 2 degrees C. below T2, the RTC 38 is enabled and the ESP 46 is powered up (wakes) and temperature is once again recorded and stored until the temperature returns to T1 or lower at which point the ESP 46 is powered down.

Finally when the temperature reached T4, such as 40 degrees C., it wakes up the ESP 46 and the ESP 46 checks for Wi-Fi to scan for SSID locations and stores the location in STORAGE 58 and then enables the MODEM 26 to check in and transmit all the temperature and location data.

WAKE 54 is also monitoring time via the RTC 38. When the RTC 38 hits its scheduled alarm, it wakes up the ESP 43. The ESP 46 then scans the Wi-Fi network, stores the network information in STORAGE 58 and then wakes up the MODEM 26 (with eSim 27) to call home and transmit the location data.

As far as data/notifications, the device 65 detects the status of the washing/sterilization of the medical instrument kit 12 by the autoclave and can provide a notification that the medical device instrument kit 12 is ready to be picked up. More specifically, after the device 65 of the present invention detects an autoclave or a wash profile, and the rigid-flex circuit assembly 10 has reached a safe temperature for Zone 3 to operate, it transmits the temperature and location data to the cloud. Once the temperature data is received in the portal, the portal shows the wash or autoclave event and sends notifications to predetermined users that an event has occurred. Once in the portal, a second algorithm is run to determine if a surgical event has occurred based on washes and autoclaves in a specific location. A surgical event is determined by an autoclave, which is required prior to surgery, followed by a wash or decontamination cycle, which is required post-surgery, in the same physical location, such as a hospital. Therefore, surgical event information can be used to determine usage of instrument kits 12 and in instances where items in the instrument kit 12 require calibration after a specific number of surgeries.

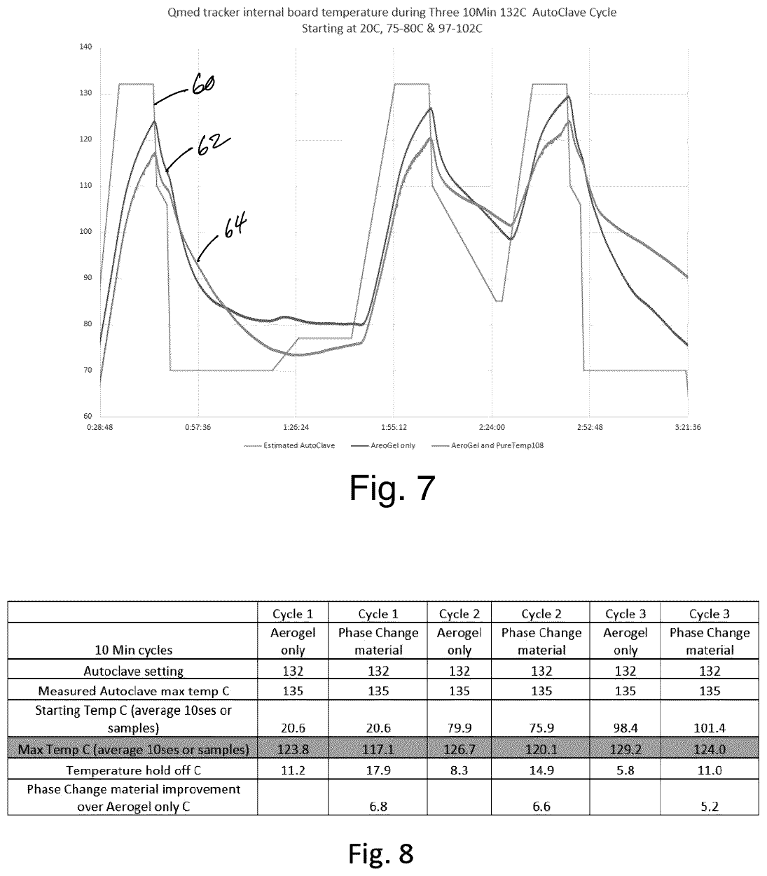

Test results are shown in FIGS. 7 and 8 for different cycles. In FIG. 7, the internal board temperature is shown over time, namely during three 10 minute autoclave cycles at 132 degrees C. and starting at 20 degrees C., 75-80 degrees C. and 97-102 degrees C. Curves for estimated sensed temperature 60 of the rigid-flex circuit assembly 10 when being sterilized in an autoclave with additional insulative material: 1) Aerogel only (an insulative material) 62, 2) Aerogel and phase change 64 materials. The results show that phase change material is helpful in cooling the rigid-flex circuit assembly 10 several degrees C., as shown in the last row of the table of FIG. 8.

It will be appreciated by those skilled in the art that various changes and modifications can be made to the illustrated embodiments without departing from the spirit of the present disclosure. All such modifications and changes are intended to be covered by the appended claims.

What is claimed is:

1. A tracking system for a medical instrument kit, comprising:
a housing; and
an electronics module contained within the housing, the electronics module including:
a power supply;
a first power zone including a linear regulator, temperature sensor, real-time clock; the first zone being always on and powered by the power supply;
a processor power zone including a DC/DC converter, microprocessor, and memory; the processor power zone being controlled by the first power zone; the first zone turns on the microprocessor at a specified time and/or at a specified temperature;
a modem power zone including a DC/DC converter and modem; the modem power zone being controlled by the processor zone when a wireless communication is required;
wherein the tracking system is configured and arranged to withstand an autoclave sterilization process.

2. The tracking system of claim 1, wherein the modem is a cellular modem.

3. The tracking system of claim 1, wherein the autoclave process includes temperatures up to approximately 132 degrees C. and maximally up to approximately 150 degrees C. and steam pressure of approximately 28 psi and maximally up to approximately 33 psi.

4. The tracking system of claim 1, wherein the power supply is a battery.

5. The tracking system of claim 1, wherein the microprocessor is an event stream processor.

6. The tracking system of claim 1, wherein the tracking system is affixed to a medical instrument kit, the medical instrument kit being configured and arranged to receive at least one medical instrument.

7. The tracking system of claim 1, wherein the tracking system resides inside a medical instrument kit, the medical instrument kit being configured and arranged to receive at least one medical instrument.

8. A method of tracking a medical instrument kit, comprising:
providing a housing; and
providing an electronics module contained within the housing, the electronics module including:
a power supply;
a first power zone including a linear regulator, temperature sensor, real-time clock;
a processor power zone including a DC/DC converter, microprocessor, and memory;
a modem power zone including a DC/DC converter and modem;
maintaining constant power from the power supply to the first zone;
controlling the processor power zone by the first power zone; turning on the microprocessor by the first zone at a specified time and/or at a specified temperature;
controlling the modem power zone by the processor zone when a wireless communication is required;
wherein the tracking system is configured and arranged to withstand an autoclave sterilization process.

9. The method of claim 8, wherein the modem is a cellular modem.

10. The method of claim 8, wherein the autoclave process includes temperatures up to approximately 132 degrees C. and maximally up to approximately 150 degrees C. and steam pressure of approximately 28 psi and maximally up to approximately 33 psi.

11. The method of claim 8, wherein the power supply is a battery.

12. The method of claim 8, wherein the microprocessor is an event stream processor.

13. The method of claim 8, wherein the tracking system is affixed to a medical instrument kit, the medical instrument kit being configured and arranged to receive at least one medical instrument.

14. The method of claim 8, wherein the tracking system resides inside a medical instrument kit, the medical instrument kit being configured and arranged to receive at least one medical instrument.
